(19) United States
(12) Patent Application Publication
(10) Pub. No.: US 2026/0294280 A1
(43) Pub. Date: Oct. 1, 2026

(54) HEART FAILURE RISK USING MECHANICAL ACTIVITY

(71) Applicant: Medtronic, Inc., Minneapolis, MN (US)

(72) Inventor: Subham Ghosh, Blaine, MN (US)

(21) Appl. No.: 19/477,564

(22) PCT Filed: Apr. 25, 2024
(86) PCT No.: PCT/US2024/026343

§ 371 Date: Oct. 22, 2025

Related U.S. Application Data
(60) Provisional application No. 63/462,080, filed on Apr. 26, 2023.

(51) Int. Cl. A61B 5/11; A61B 5/00; A61B 7/04

(52) U.S. Cl. CPC A61B 5/1126; A61B 5/7264; A61B 5/7275; A61B 7/04; A61B 5/686; A61B 5/746

(57) ABSTRACT

A heart failure risk score may be determined using one or more heart failure metrics. Each of the heart failure metrics may be generated, or determined, based on, at least, mechanical activity of a patient's heart monitored using a mechanical heart activity sensor. The mechanical heart activity sensor may include one or more of a piezoelectric sensor to monitor vibration of the heart, a motion sensor to monitor heart motion, and a microphone to monitor heart sounds.

### RELATED APPLICATIONS

This application claims priority to U.S. Provisional Patent Application No. 63/462,080, filed on Apr. 26, 2023, which is incorporated by reference herein in its entirety.

This disclosure generally relates to determination of a heart failure risk using mechanical activity.

Chronic heart failure (HF) occurs when a heart of a patient is unable to consistently pump blood at an adequate rate in response to the filling pressure. To improve the ability of the heart to pump blood, congestive heart failure patients may be given therapy. In some cases, therapy may be provided by implantable medical devices (IMDs) such as implantable cardioverter defibrillators (ICDs), cardiovascular implantable electronic devices (CIEDs), pacemakers, and cardiac resynchronization therapy (CRT) devices that, in some cases, include defibrillation capability (CRT-D devices).

Despite using IMDs to improve heart function, some HF patients may experience worsening HF. Some systems are capable of identifying patients at risk of worsening HF, for example, based on data detected by IMDs. One exemplary system relates to U.S. Pat. No. 9,713,701 to Sarkar et al. that is capable of generating alerts for a patient to seek medical treatment in response to detected information. For example, a medical device may detect worsening heart failure in the patient based on a diagnostic parameter. Upon detecting worsening heart failure, the medical device may, for example, provide an alert. Once patients at risk are identified with an alert, some patients may benefit from hospitalization, while others may benefit from specific interventions. Some existing systems can provide guidance using diuretics. Effective actions guided by diagnostics have been difficult to determine to provide more complete HF management.

### SUMMARY

This disclosure generally relates to determination a heart failure risk score based on one or more (e.g., a plurality of) heart failure metrics using, among other things, monitored mechanical activity of a patient's heart. The determination of heart failure risk score described herein may be described as being able to help assess, or assist in the assessment of, heart failure risk and to help device-based heart failure management.

The mechanical activity of the patient's heart may be measured, or monitored, using one or more mechanical heart activity sensors such as, for example, a piezoelectric sensor to monitor vibrations of the heart, a motion sensor (e.g., accelerometer) to monitor heart motion, and a microphone to monitor heart sounds. The one or more mechanical heart activity sensors may be part of, or included in, an implantable medical device (IMD) such as, for example, implantable cardioverter defibrillators (ICDs), cardiovascular implantable electronic devices (CIEDs), pacemakers, and cardiac resynchronization therapy (CRT) devices that, in some cases, include defibrillation capability (CRT-D). The heart failure risk score may be described as being determined, or generated, based on mechanical activity of the patient's heart in conjunction with electrical activity of the patient's heart. For example, an electromechanical activation time interval may be determined between a monitored electrical activation event and a monitored mechanical event, which may be used to determine, or generate, a heart failure risk score.

The illustrative systems, devices, and methods may be described as being able to extract components from valid heart sounds that contain diagnostic information on cardiac function, and then determine a heart failure risk score using such extracted components from the valid heart sounds. The extracted components or information determined, or generated, therefrom may be referred to as heart failure metrics. In one or more embodiments, the extracted components of the heart sounds may include one or more aspects or components of the first heart sound also referred to as S1, the second heart sound also referred to as S2, the third heart sound also referred to as S3, and the fourth heart sound also referred to as S4, as will be described further herein. In particular, the extracted components of the heart sounds may include one or more of an early cardiac cycle mechanical maximum amplitude corresponding to the first heart sound, S1, a time of the cardiac cycle mechanical amplitude corresponding to the first heart sound, S1, to relative to an onset of an intrinsic ventricular sensing or ventricular/biventricular pacing event, a late cycle mechanical maximum amplitude corresponding to one or both of the third and fourth heart sounds, S3 and S4, and a ratio of the late cardiac cycle mechanical maximum amplitude and the early cardiac cycle mechanical maximum amplitude.

In one embodiment, the illustrative systems, devices, and methods may be involved in one or more of defining a signal of interest within the heart sound signal detected, or monitored, by a piezoelectric sensor or a accelerometer sensor between two successive ventricular events as indicated by implantable device sensing/pacing, determining a peak of the signal within first 40% of the cycle-length as defined by the two successive ventricular events sensed by the device, determining a S1 amplitude as the difference between this peak and the baseline amplitude (such as, for example, 0), and determining the time from beginning of the first ventricular event to the time when the signal first crosses a threshold of the S1 amplitude (for example, 70% of the S1 amplitude), which may be referred to as an electromechanical interval or the timing of S1 event. Additionally, the illustrative systems, devices, and methods may be involved in one or more of determining the max amplitude of the heart sounds signal within the last 20% of the cycle length and finding the difference between the max amplitude in the last 20% of the cycle length and a baseline amplitude (such as, for example, 0) as the amplitude of the late-cycle sounds, which may be referred to as a S3/S4 composite amplitude or late maximum value. Further, the illustrative systems, devices, and methods may be involved in determining the amplitude ratio of the S3/S4 composite amplitude or late maximum value to the S1 amplitude, which may be referred to as the early/late ratio. The illustrative systems, devices, and methods may monitor, or record, the heart sound signals for a plurality of cardiac cycles and compute averages, medians, or other statistical measures of the one or more extracted components or heart failure metrics over the plurality of cardiac cycles.

A heart failure risk score may be generated based on the one or more extracted components or heart failure metrics. In one embodiment, the heart failure risk score may be initialized to, or started at, 0. Next, if the electromechanical interval or the timing of S1 event is less than a lower electromechanical activation threshold such as, for example, 170 milliseconds (ms), or greater than an upper electromechanical activation threshold, such as, for example, 400 ms, then heart failure risk score may be increased by 1. Further, if the S3/S4 composite is greater than a late cycle threshold such as, for example, 89 millivolts (mV), then the heart failure risk score may be increased by 1. Still further, if the early/late ratio is greater than exceeds an early/late ratio threshold such as, for example, 0.5, then the heart failure risk score may be increased by 1. The heart failure risk score may be updated periodically such as, for example, daily, and presented to a patient via a display, uploaded to patient care management system, and present to a clinician. In one embodiment, the heart failure risk score may be plotted over time on graph or plot so as to indicate or display a trend. If the heart failure risk score continues to be greater than or equal to 2 for a selected number of consecutive, or successive, days, then the patient may be determined to be at high risk of heart failure.

One illustrative device may include a mechanical heart activity sensor configured to monitor mechanical activity of a patient's heart and a computing apparatus comprising processing circuitry and operably coupled to the mechanical heart activity sensor. The computing apparatus may be configured to monitor mechanical activity of the patient's heart using the mechanical heart activity sensor, determine a plurality of heart failure metrics for one or more cardiac cycles using the monitored mechanical activity, and determine a heart failure risk score based on the determined plurality of heart failure metrics for the one or more cardiac cycles.

One illustrative method may include monitoring mechanical activity of a patient's heart, determining a plurality of heart failure metrics for one or more cardiac cycles using the monitored mechanical activity, and determining a heart failure risk score based on the determined plurality of heart failure metrics for the one or more cardiac cycles.

One illustrative system may include a remote computing apparatus comprising processing circuitry. The remote computing apparatus may be configured to receive mechanical heart activity of a patient and determine a plurality of heart failure metrics for one or more cardiac cycles using received mechanical heart activity, and determine a heart failure risk score based on the determined plurality of heart failure metrics for the one or more cardiac cycles.

The plurality of heart failure metrics comprises an electromechanical activation time interval, a late maximum value, and an early/late ratio between the late maximum value and an early maximum value. Determining the plurality of heart failure metrics for each of the one or more cardiac cycles may include determining the early maximum value of the mechanical activity within an early monitoring window following a first ventricular electrical activation event, determining the electromechanical activation time interval based on the first ventricular electrical activation event and the early maximum value, determining the late maximum value of the mechanical activity within a late monitoring window preceding a second ventricular electrical activation event following the first ventricular electrical activation event, and determining the early/late ratio between of the late maximum value and the early maximum value.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a diagram of an illustrative system including an implantable medical device (IMD) and a programmer.

FIG. 2 is a diagram of the illustrative IMD of FIG. 1.

FIG. 3 is a block diagram of the IMD of FIGS. 1-2.

FIG. 4 is a diagram of an illustrative programmer of the system of FIG. 1.

FIG. 5 is a diagram of an illustrative system including the IMD and programmer of FIG. 1 and additional devices coupled thereto via a network.

FIG. 6 is an illustrative method of determining heart failure risk, e.g., using the system and devices of FIGS. 1-5.

FIG. 7 is an illustrative method of determining a composite heart failure risk metric.

FIG. 8 is an illustrative graph of a mechanical heart activity signal over two cardiac cycles illustrating heart failure metrics for use with the systems, devices, and methods of FIGS. 1-7 and 9.

FIG. 9 is an illustrative method of determining heart failure risk metrics and heart failure risk score of FIG. 6.

FIG. 10 is a comparative bar graph showing the correlation between electromechanical interval being indicative of increased heart failure risk and patients that experienced heart failure events and for patients that did not experience heart failure events.

FIG. 11 is a comparative bar graph showing the correlation between late maximum value of mechanical activity being indicative of increased heart failure risk and patients that experienced heart failure events and for patients that did not experience heart failure events.

FIG. 12 is a comparative bar graph showing the correlation between early/late ratio of mechanical activity being indicative of increased heart failure risk and patients that experienced heart failure events and for patients that did not experience heart failure events.

FIG. 13 is a comparative bar graph showing the correlation between heart risk score for patients being indicative of increased heart failure risk and patients that experienced heart failure events and for patients that did not experience heart failure events.

FIG. 14 is a comparative bar graph showing the correlation between early maximum value, or S1 amplitude, being indicative of increased heart failure risk and patients that experienced heart failure events and for patients that did not experience heart failure events. early/late ratio of mechanical activity.

### DETAILED DESCRIPTION

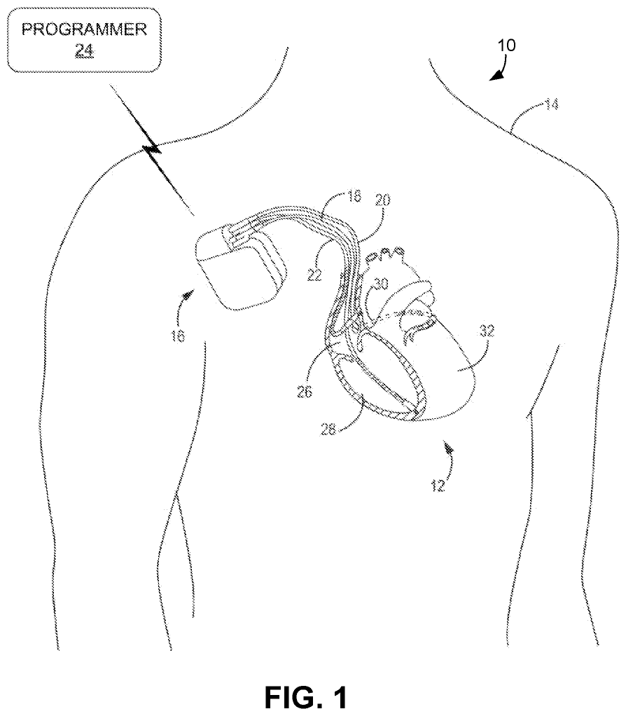

The techniques of this disclosure generally relate to determination of a heart failure risk score using monitored, or measured, mechanical heart activity. The heart failure risk score may be measured and determined using implantable medical devices (IMDs). Illustrative systems, devices, methods, and processes that are used to determine heart failure risk score are described herein with respect to FIGS. 1-14.

FIG. 1 is a conceptual diagram of an exemplary therapy system 10 that may be used to monitor mechanical heart activity of a patient 14, determine one or more heart failure metrics, and determine a heart failure risk score. Additionally, the system 10 may be configured to deliver pacing therapy, such as cardiac pacing therapy and cardiac resynchronization therapy, to a patient 14. While the patient 14 is shown as a human, the patient 14 may also be a variety of other types of animals. The therapy system 10 may include an implantable medical device 16 (IMD), which may be coupled to leads 18, 20, 22, and a programmer 24. The IMD 16 may be, e.g., an implantable pacemaker, cardioverter, and/or defibrillator, that senses mechanical activity (e.g., sounds, motions, vibrations, etc.) of the heart 12 of the patient 14 via a mechanical heart activity sensor, delivers, or provides, electrical signals (e.g., paces, etc.) to the heart 12 of the patient 14 via electrodes coupled to one or more of the leads 18, 20, 22, and/or senses electrical signals from the heart 12 of the patient 14 via electrodes coupled to one or more of the leads 18, 20, 22.

The leads 18, 20, 22 extend into the heart 12 of the patient 14 to sense electrical activity of the heart 12 and/or to deliver electrical stimulation to the heart 12. In the example shown in FIG. 1, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, mechanical heart activity signals of the heart 12 using a mechanical heart activity sensor and electrical signals attendant to the depolarization and repolarization of the heart 12 via electrodes coupled to at least one of the leads 18, 20, 22. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., pacing rate, R-R interval, A-V delay and other various timings, pulse width, amplitude, voltage, burst length, etc. Further, the IMD 16 may be operable to use various electrode configurations to deliver pacing therapy, which may be unipolar, bipolar, quadripolar, or further multipolar. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD, or electrode apparatus. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the IMD 16 may detect arrhythmia of the heart 12, such as fibrillation of the ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of the heart 12 is stopped.

In some examples, the programmer 24 may be a mobile computing device (such as a smartphone) or a computer workstation. The programmer 24 may include a user interface that receives input from a user. The user interface may include, for example, a keypad and a display, which may, for example, be a liquid crystal display (LCD) or light emitting diode (LED) display. The keypad may take the form of an alphanumeric keypad or a reduced set of keys associated with particular functions. The programmer 24 can additionally or alternatively include a peripheral pointing device, such as a mouse, via which a user may interact with the user interface. In some embodiments, a display of the programmer 24 may include a touch screen display, and a user may interact with the programmer 24 via the display.

A user, such as a physician, technician, patient, or other user, may interact with the programmer 24 to communicate with the IMD 16. For example, a user may interact with the programmer 24 to retrieve physiological or diagnostic information from the IMD 16. A user may also interact with the programmer 24 to program the IMD 16, e.g., select values for operational parameters of the IMD.

Further, for example, a user may use the programmer 24 to retrieve information from IMD 16 regarding one or more heart failure metrics, heart failure risk score, trends related one or more heart failure metrics, heart failure risk score, heart failure risk alerts, rhythm of the heart 12, trends over time, or tachyarrhythmia episodes. As another example, a user may use the programmer 24 to retrieve information from the IMD 16 regarding other sensed physiological or diagnostic parameters of the patient 14, such as intracardiac or intravascular pressure, activity, posture, respiration, or thoracic impedance. As another example, the user may use the programmer 24 to retrieve information from the IMD 16 regarding the performance or integrity of the IMD 16 or other components of the system 10, such as the leads 18, 20, and 22, or a power source of the IMD 16.

A user may use the programmer 24 to review one or more heart failure metrics, heart failure risk score, and trends related thereto. In some examples, a user may activate features of the IMD 16 by entering a single command via the programmer 24, such as depression of a single key or combination of keys of a keypad or a single point-and-select action with a pointing device.

The IMD 16 and the programmer 24 may communicate via wireless communication using any techniques known in the art. Examples of communication techniques may include, for example, low frequency or radiofrequency (RF) telemetry, but other techniques are also contemplated. In some examples, the programmer 24 may include a programming head that may be placed proximate to the patient's body near the IMD 16 implant site in order to improve the quality or security of communication between the IMD 16 and the programmer 24.

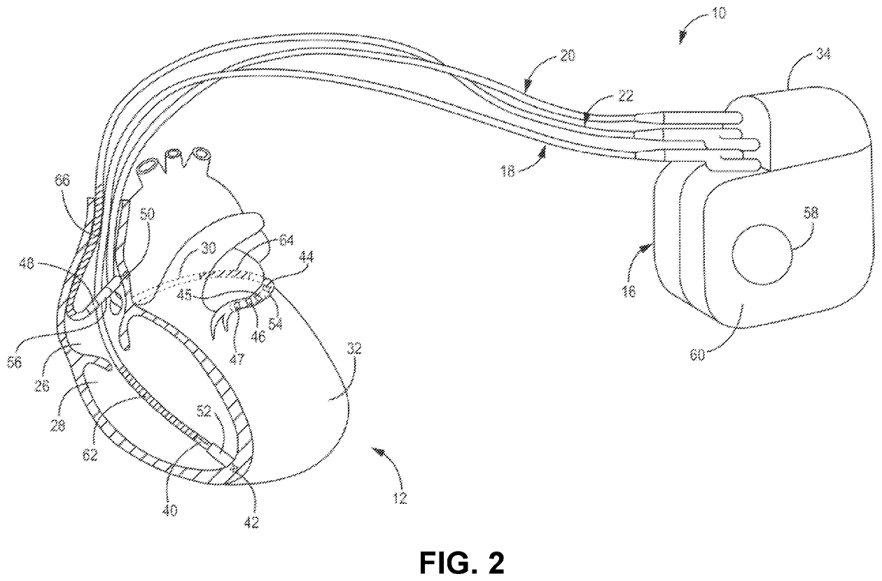

FIG. 2 is a conceptual diagram of the IMD 16 and the leads 18, 20, 22 of therapy system 10 of FIG. 1 in more detail. The leads 18, 20, 22 may be electrically coupled to a therapy delivery module (e.g., for delivery of cardiac remodeling pacing therapy), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the IMD 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry a number of conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of, or define, ring electrodes, and the electrodes 42, 50 may take the form of, or define, extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient's heart 12. In some examples, as illustrated in FIG. 2, the IMD 16 includes one or more housing electrodes, such as housing electrode 58, which may be formed integrally with an outer surface of a housing 60 (e.g., hermetically sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 2, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient's heart during the delivery of pacing therapy (e.g., in combination with the housing electrode 58, or defibrillation electrode-to-housing electrode vector).

The above-described configuration of the therapy system 10 is merely one example. In other examples, the therapy system may include epicardial leads and/or patch electrodes instead of, or in addition to, the transvenous leads 18, 20, 22 illustrated in FIG. 1. In further embodiments, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 1). Further, in one or more embodiments, the IMD 16 may not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

Other example therapy systems that provide electrical stimulation therapy to the heart 12 may include any suitable number of leads coupled to the IMD 16, and each of the leads may extend to any location within or proximate to the heart 12. Such other therapy systems may include three transvenous leads located as illustrated in FIGS. 1-2. Still further therapy systems may include a single lead that extends from the IMD 16 into the right atrium 26 or two leads that extend into a respective one of the right atrium 26 and the left atrium. In one example, the IMD 16, as a cardiac resynchronization therapy (CRT) device with a left ventricular (LV) lead, may be useful for a HFpEF patient if there is a complete AV node block as a LV lead can be more beneficial than a RV lead in such patients.

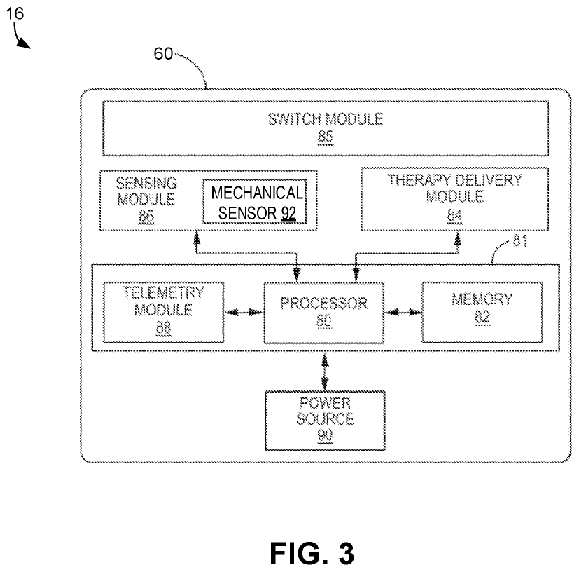

FIG. 3 is a functional block diagram of an illustrative configuration of the IMD 16. As shown, the IMD 16 may include a control module 81, a therapy delivery module 84 (e.g., which may include a stimulation generator), a sensing module 86, and a power source 90. The control module, or apparatus, 81 may include a computing apparatus 80, memory 82, and a telemetry module, or apparatus, 88. The memory 82 may include computer-readable instructions that, when executed, e.g., by the computing apparatus 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically erasable programmable ROM (EEPROM), flash memory, and/or any other digital media.

The computing apparatus 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the computing apparatus 80 may include multiple components, such as any combination of one or more microprocessors, one or more controllers, one or more DSPs, one or more ASICs, and/or one or more FPGAs, as well as other discrete or integrated logic circuitry. The functions attributed to the computing apparatus 80 herein may be embodied as software, firmware, hardware, or any combination thereof.

The control module 81 may be configured to perform one or more methods and processes described herein within respect to generation, or determination, of one or more heart failure metrics and a heart failure risk score and processing of a mechanical heart activity signal received from, or monitored by, a mechanical heart activity sensor. Further, the control module 81 may control the therapy delivery module, or apparatus, 84 to deliver therapy (e.g., cardiac resynchronization therapy, adaptive pacing, bradycardia pacing, etc.) to the heart 12 according to a selected one or more therapy programs, which may be stored in the memory 82, and based on algorithms, or methods, described further below. More specifically, the control module 81 (e.g., the computing apparatus 80) may control various parameters of the electrical stimulus delivered by the therapy delivery module 84 such as, e.g., A-V delays, pacing pulses with the amplitudes, pulse widths, frequency, or electrode polarities, etc., which may be specified by one or more selected therapy programs (e.g., adaptive pacing therapy program, lower pacing rate limit determination, adjustment, and/or modifications programs, A-V delay adjustment programs, pacing therapy programs, pacing recovery programs, capture management programs, etc.). Additionally, the control module 81 may control such described therapy in response to, or based on, the determined one or more heart failure metrics and heart failure risk score. As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, the therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to the heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured to deliver one or more of these types of stimulation in the form of other signals, such as sine waves, square waves, and/or other substantially continuous time signals.

The IMD 16 may further include a switch module, or apparatus, 85 and the control module 81 (e.g., the computing apparatus 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module, or apparatus, 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switch module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave alternans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc.

The sensing module 86 may further include a mechanical heart activity sensor 92 configured to monitor mechanical activity of the patient's heart 12. The mechanical activity of the patient's heart may include or be representative of one or more motion, movement, sounds, and vibrations of the patient's heart 12 and one or more portions or anatomical mechanisms of the patient's heart 12. For example, the mechanical heart activity sensor 92 may be configured to monitor mechanical activity corresponding to or indicative of closure of the atrioventricular valves (i.e., the mitral valve and the tricuspid valve), closure of the semilunar valves (i.e., the aortic valve and the pulmonary valve), chamber fillings, chamber contractions, transitions from rapid to slow filling, low frequency vibrations at early diastole that may be indicative of systolic heart failure, and low-pitched sounds at late diastole arising from atrial contraction. In particular, for example, the mechanical heart activity sensor 92 may be configured to monitor mechanical activity corresponding to or indicative of the heart sound S1 that may be described as representing closure of the atrioventricular valves (i.e., the mitral valve and the tricuspid valve) as the ventricular pressures exceed the atrial pressures at the beginning of systole. The heart sound S1 may typically be a single sound as the mitral and tricuspid valves close nearly simultaneously. Further, for example, the mechanical heart activity sensor 92 may be configured to monitor mechanical activity corresponding to or indicative of the heart sound S2 that may be described as representing represents closure of the semilunar valves (i.e., the aortic valve and the pulmonary valve). Still further, for example, the mechanical heart activity sensor 92 may be configured to monitor mechanical activity corresponding to or indicative of the heart sounds S3 and S4 that may be described as representing a transition from rapid to slow ventricular filling in early diastole and as an abnormal late diastolic sound caused by forcible atrial contraction in the presence of decreased ventricular compliance, respectively.

It is to be understood that, although the mechanical heart activity sensor 92 is depicted as being part of the sensing module 86 within the housing 60 of the IMD 16, the mechanical heart activity sensor 92 may be external to the housing 60 such as, e.g., part of or included within one of the leads 18, 20, 22, and positioned in various locations within or about the patient's heart 12. Further, in at least one embodiment, the IMD 16 may be a leadless IMD including the mechanical heart activity sensor 92 therein and may be positioned within a chamber of the patient's heart 12 thereby placing the mechanical heart activity sensor 92 within the chamber of the patient's heart 12.

The mechanical heart activity sensor 92 may include one or more of a piezoelectric sensor to monitor vibration of the heart, a motion sensor to monitor heart motion, and a microphone to monitor heart sounds. The mechanical heart activity sensor 92 may be used, as described further herein, to determine or generate one or more heart failure metrics and heart failure risk score.

In various embodiments, the mechanical heart activity sensor 92 may be described as a heart sound (HS) sensor and may be implemented as a microphone and/or a 1-, 2- or 3-axis accelerometer. In one embodiment, the mechanical heart activity sensor 92 is implemented as a piezoelectric crystal mounted that is responsive to the mechanical motion associated with heart sounds. Examples of other embodiments of mechanical heart activity sensors that may be adapted for implementation with the techniques of the present disclosure may be described generally in U.S. Pat. Nos. 4,546,777, 6,869,404, 5,554,177, and 7,035,684, each of which is incorporated herein by reference in its entirety. Moreover, for example, the mechanical heart activity sensor 92 may include any suitable transducer components (e.g., mounted within the implanted device, mounted on the can of the device, etc.) for sensing valve activity, such as a sonomicrometer, an accelerometer, a cardiomechanical sensor (CMES) employing embedded piezoelectric material on an implanted lead or alternate piezoelectric sensor. In other embodiments, heart valve events, such as mitral valve closure and aortic valve closure, may be detected using non-acoustic sensors, including, for example, sensors embedded in the myocardium or pressure sensors implanted to detect chamber pressures, etc. Such detected valve events (e.g., heart sounds) may be used to provide information for one or more functions, including those described herein.

In other words, various sensor types may be used as the mechanical heart activity sensor 92. For example, the mechanical heart activity sensor 92 may be described as being any implantable or external sensor responsive to one or more of the mechanical heart activity, and thereby, capable of producing, or generating, an electrical analog signal correlated in time and amplitude to the mechanical heart activity. The analog signal may then be processed, which may include digital conversion, by the sensing module 86 to obtain mechanical activity in the form of a mechanical activity signal, of which various metrics such as amplitudes or relative time intervals may be derived or generated.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 40, 42, 44, 45, 46, 47, 48, 50, 58, 62, 64, 66), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any mathematical calculations may be performed by the computing apparatus 80 and any updating of the values or intervals controlled by the pacer timing and control module may be executed, or take place, following such interrupts. A portion of memory 82 may be configured as a plurality of recirculating buffers, capable of holding one or more series of measured intervals, which may be analyzed by, e.g., the computing apparatus 80 in response to the occurrence of a pace or sense interrupt to determine whether the patient's heart 12 is presently exhibiting atrial or ventricular tachyarrhythmia.

Further, the computing apparatus 80 of IMD 16 may detect a tachyarrhythmia episode, such as a ventricular fibrillation, ventricular tachycardia, fast ventricular tachyarrhythmia episode, or a NST episode, based on electrocardiographic activity of heart 12 that is monitored via sensing module 86. For example, sensing module 86, with the aid of at least some of the electrodes 40, 42, 44, 46, 48, 50, 58, 62, 64, and 66 (shown in FIGS. 1-2), may generate an electrocardiogram (ECG) or electrogram (EGM) signal that indicates the electrocardiographic activity, sensing module 86 may be coupled to sense electrodes that are separate from the stimulation electrodes that deliver electrical stimulation to heart 12 (shown in FIGS. 1-2), and may be coupled to one or more different leads than leads 18, 20, 22 (shown in FIGS. 1-2). The ECG signal may be indicative of the depolarization of heart 12.

For example, as previously described, in some examples, computing apparatus, or processor, 80 may identify the presence of a tachyarrhythmia episode by detecting a threshold number of tachyarrhythmia events (e.g., R-R or P-P intervals having a duration less than or equal to a threshold). In some examples, the computing apparatus 80 may also identify the presence of the tachyarrhythmia episode by detecting a variable coupling interval between the R-waves of the heart signal.

The telemetry module 88 of the control module 81 may include any suitable hardware, firmware, software, or any combination thereof for communicating with another device, such as a programmer (such as, for example, a mobile computing devices or smartphone). For example, under the control of the computing apparatus 80, the telemetry module 88 may receive downlink telemetry from and send uplink telemetry to a programmer or mobile computing device with the aid of an antenna, which may be internal and/or external. The computing apparatus 80 may provide the data to be uplinked to a programmer or a mobile computing device and the control signals for the telemetry circuit within the telemetry module 88, e.g., via an address/data bus. In some examples, the telemetry module 88 may provide received data to the computing apparatus 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

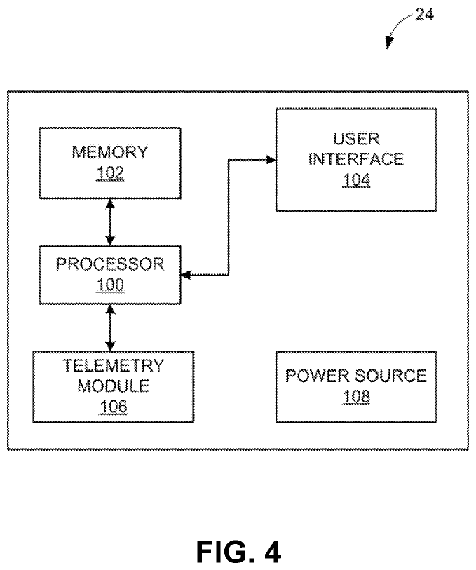

FIG. 4 is a block diagram of an illustrative programmer 24. As shown in FIG. 4, the programmer 24 includes a processor 100, a memory 102, a user interface 104, a telemetry module 106, and a power source 108. The programmer 24 may be a dedicated hardware device with dedicated software for programming of IMD 16. Alternatively, the programmer 24 may be an off-the-shelf computing device (e.g., mobile compute device such as a smartphone) running an application that enables programmer 24 to program IMD 16.

A user may use the programmer 24 to display and review a patient's health, one or more heart failure metrics, heart failure metric risk score, and trends related thereto. Additionally, a user may use the programmer 24 to select therapy programs (e.g., sets of stimulation parameters), generate new therapy programs, modify therapy programs through individual or global adjustments or transmit the new programs to a medical device, such as the IMD 16 of FIG. 1. A user may interact with the programmer 24 via the user interface 104, which may include display to present graphical user interface to a user, and a keypad or another mechanism for receiving input from a user.

The processor 100 can take the form one or more microprocessors, DSPs, ASICs, FPGAs, programmable logic circuitry, or the like, and the functions attributed to processor 100 herein may be embodied as hardware, firmware, software or any combination thereof. The memory 102 may store instructions that cause processor 100 to provide the functionality ascribed to the programmer 24 herein, and information used by processor 100 to provide the functionality ascribed to the programmer 24 herein. The memory 102 may include any fixed or removable magnetic, optical, or electrical media, such as RAM, ROM, CD-ROM, hard or floppy magnetic disks, EEPROM, or the like. The memory 102 may also include a removable memory portion that may be used to provide memory updates or increases in memory capacities. A removable memory may also allow IMD and/or patient data to be easily transferred to another computing device, or to be removed before the programmer 24 is used to program therapy for another patient. The memory 102 may also store information that controls therapy delivery by the IMD 16, such as stimulation parameter values.

The programmer 24 may communicate wirelessly with the IMD 16, such as using RF communication or proximal inductive interaction. This wireless communication is possible through the use of the telemetry module 106, which may be coupled to an internal antenna or an external antenna. An external antenna that is coupled to programmer 24 may correspond to the programming head that may be placed over the heart 12, as described above with reference to FIG. 1. The telemetry module 106 may be similar to telemetry module 88 of the IMD 16 of FIG. 3.

The telemetry module 106 may also be configured to communicate with another computing device via wireless communication techniques, or direct communication through a wired connection. Examples of local wireless communication techniques that may be employed to facilitate communication between the programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with the programmer 24 without needing to establish a secure wireless connection.

The power source 108 delivers operating power to the components of programmer 24 and may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within programmer 24. In other embodiments, traditional batteries (e.g., nickel cadmium or lithium-ion batteries) may be used. In addition, programmer 24 may be directly coupled to an alternating current outlet to power the programmer 24. The power source 108 may include circuitry to monitor power remaining within a battery. In this manner, a user interface 104 may provide a current battery level indicator or low battery level indicator when the battery needs to be replaced or recharged. In some cases, power source 108 may be capable of estimating the remaining time of operation using the current battery.

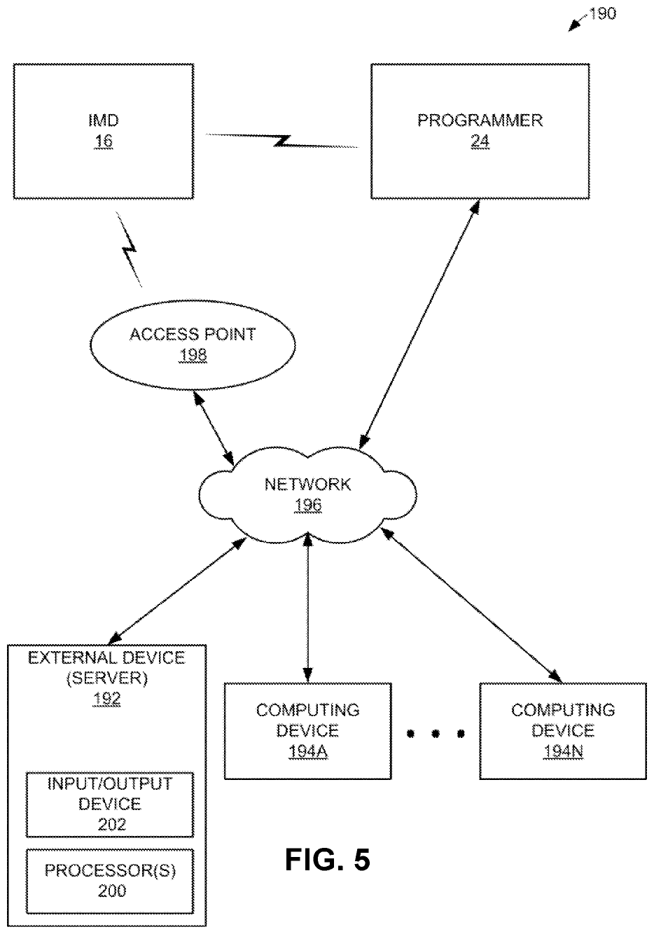

FIG. 5 is a block diagram illustrating a system 190 that includes an external device 192, such as a server, and one or more computing devices 194a-194n that are coupled to the IMD 16 and the programmer 24 shown in FIGS. 1-4 via a network 196, according to one embodiment. In this embodiment, the IMD 16 may use its telemetry module 88 to communicate with the programmer 24 via a first wireless connection, and to communicate with an access point 198 via a second wireless connection. In the example of FIG. 5, the access point 198, the programmer 24, the external device 192, and the computing devices 194a-194n are interconnected, and able to communicate with each other, through a network 196. In some cases, one or more of the access point 198, the programmer 24, the external device 192, and the computing devices 194a-194n may be coupled to the network 196 through one or more wireless connections. The IMD 16, the programmer 24, the external device 192, and the computing devices 194a-194n may each include, or comprise, one or more processors, such as one or more microprocessors, DSPs, ASICs, FPGAs, programmable logic circuitry, or the like, that may perform various functions and operations, such as those described herein.

The access point 198 may include, or comprise, a device that connects to the network 196 via any of a variety of connections, such as cellular data connection, telephone dial-up, digital subscriber line (DSL), or cable modem connections. In other examples, the access point 198 may be coupled to the network 196 through different forms of connections, including wired or wireless connections. In some examples, the access point 198 may communicate with the programmer 24 and/or the IMD 16. The access point 198 may be co-located with the patient 14 (e.g., within the same room or within the same site as the patient 14) or may be remotely located from the patient 14. For example, the access point 198 may be a home monitor that is located in the patient's home or is portable for carrying with the patient 14.

During operation, the IMD 16 may collect, measure, and store various forms of diagnostic data such as, e.g., diagnostic parameters that may be utilized by the illustrative systems, methods, and processes to determine, or generate, one or more heart failure metrics, heart failure risk score, mechanical activity, mechanical activity, signals, and one or more events within the mechanical activity and the mechanical activity signals. In certain cases, the IMD 16 may directly analyze collected diagnostic data and generate any corresponding reports or alerts. In some cases, however, the IMD 16 may send diagnostic data such as the diagnostic parameters, to the programmer 24, the access point 198, and/or the external device 192, either wirelessly or via the access point 198 and the network 196, for remote processing and analysis (e.g., to determine a heart failure risk score).

In another example, the IMD 16 may provide the external device 192 with collected diagnostic data or parameters such as, for example, one or more heart failure metrics, heart failure risk score, and monitored mechanical heart activity, via the access point 198 and the network 196. The external device 192 includes one or more the processors 200. In some cases, the external device 192 may request such data, and in some cases, the IMD 16 may automatically or periodically provide such data to the external device 192. Upon receipt of the diagnostic data via the input/output device 202, the external device 192 may be capable of analyzing the data and generating reports, alerts, or other values (e.g., a heart failure risk score).

One or more of the computing devices 194a-194n may access the diagnostic data or parameters through the network 196 for use in determining one or more heart failure metrics and heart failure risk score. In some cases, the external device 192 may display the one or more heart failure metrics, heart failure risk score, and monitored mechanical heart activity to a user via the input/output device 202.

In one embodiment, the external device 192 may comprise a secure storage site for diagnostic data or information that has been collected from the IMD 16 and/or the programmer 24. In this embodiment, the network 196 may comprise an Internet network, and trained professionals, such as clinicians, may use the computing devices 194a-194n to securely access stored diagnostic data or parameters such as one or more heart failure metrics, heart failure risk score, and monitored mechanical heart activity on the external device 192. For example, the trained professionals may utilize secure usernames and passwords to access the stored information on the external device 192. In one embodiment, the external device 192 may be a CareLink server provided by Medtronic, Inc., of Minneapolis, Minnesota.

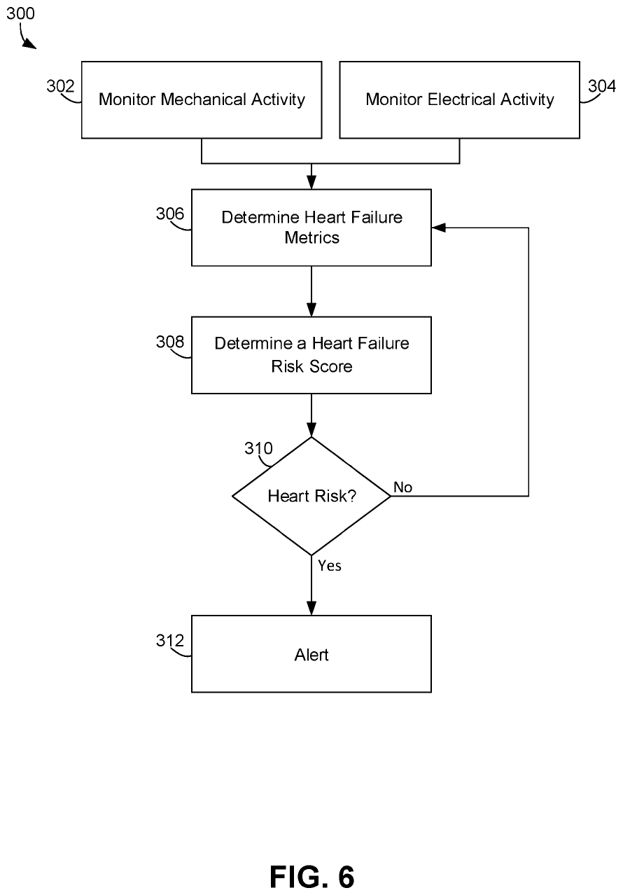

An illustrative method 300 of determining heart failure risk, e.g., using the system and devices of FIGS. 1-5, is depicted in FIG. 6. As shown, the method 300 includes monitoring mechanical activity 302 of the patient's heart using a mechanical heart activity sensor and monitoring electrical activity 304 of the patient's heart using one or more electrodes (e.g., implantable electrodes). Monitoring mechanical activity 302 may provide one or more mechanical activity signals corresponding to the mechanical activity (e.g., movement, motion, sound, and vibration) of the patient's heart and monitoring electrical activity 304 may provide one or more electrical activity signals corresponding to electrical activity of the patient's heart. Such mechanical and electrical activity signals may be used by the method 300, and other methods and processes described herein, to determine, or generate, one or more heart failure metrics and a heart failure risk score. Monitoring mechanical activity 302, such as heart sounds, may be described in U.S. Provisional Pat. App. Ser. No. 63/387,466 entitled “Determining Validity of Heart Sounds Signals from Implantable Medical Device,” filed on Dec. 12, 2022, and corresponding to Docket No.: A0009455US01/2222-333USP1, which is incorporated herein by reference in its entirety.

It is be understood that the mechanical activity and electrical activity may be monitored 302, 304 continuously, periodically for selected periods of time, in response to another initiating, or triggering, event, or intermittently according to a schedule. Additionally, the mechanical activity and electrical activity may be monitored 302, 304 for a selected period of time or selected number of cardiac cycles to provide an appropriate amount to data to determine, or generate, one or more heart failure metrics and a heart failure risk score therefrom. In one or more embodiments, the mechanical activity and electrical activity may be monitored 302, 304 between 1 cardiac cycle and about 30 cardiac cycles. In one embodiment, the mechanical activity and electrical activity may be monitored 302, 304 monitored for 5 cardiac cycles. Further, for example, the mechanical activity and electrical activity may be monitored 302, 304 for greater than or equal to 1 cardiac cycle, greater than or equal to 2 cardiac cycles, greater than or equal to 3 cardiac cycles, greater than or equal to 5 cardiac cycles, greater than or equal to 10 cardiac cycles, or greater than or equal to 15 cardiac cycles and/or less than or equal to 30 cardiac cycles, less than or equal to 25 cardiac cycles, less than or equal to 20 cardiac cycles, less than or equal to 12 cardiac cycles, or less than or equal to 8 cardiac cycles.

The method 300 may further include determining one or more heart failure metrics 306 for one or more cardiac cycles or a plurality of cardiac cycles using, or based on, the monitored mechanical activity and electrical activity. Each heart failure metric may be described as a single-cycle heart failure metric because each heart failure metric may be derived, or generated, from data, or signals, from within a single cardiac cycle. However, when determining heart failure metrics 306 for, or over, a plurality of cardiac cycles, the heart failure metrics may be generated, or determined, for each cardiac cycle of the plurality of cardiac cycles, and then composite heart failure metrics may be generated, or determined, for each heart failure metric based on the plurality of corresponding heart failure metrics generated over the plurality of cardiac cycles. In particular, a statistical metric may be utilized such as an average or median of the plurality of corresponding heart failure metrics generated over the plurality of cardiac cycles to generate, or determine, the composite heart failure metric.

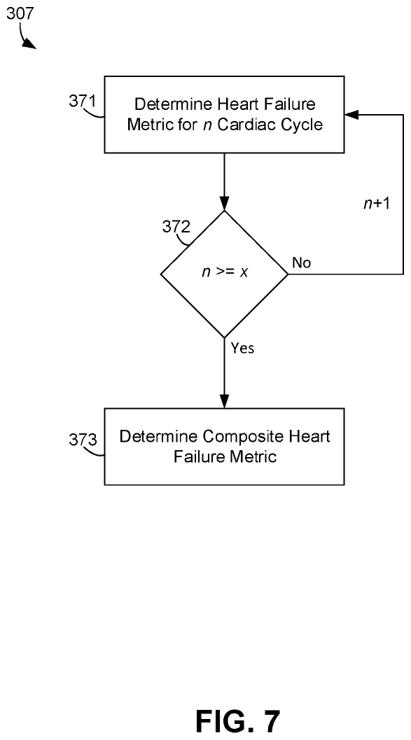

For example, an illustrative method 307 of determining a composite heart failure risk metric is depicted in FIG. 7. As shown, method 306 may determine a heart failure metric 371 for a cardiac cycle n for a plurality of cardiac cycles, x, 372. In particular, a heart failure metric may be determined 371, and then, if n, which represents the number of cardiac cycles for which the heart failure metric has been generated for, is not greater than or equal to x, which represents the number of cardiac cycles for which the heart failure metric should be generated for, then the method 300 will return to determining the heart failure metric 371 for another cardiac cycle n. If n, is greater than or equal to x, then the method 300 will stop determining the heart failure metric 371 for another cardiac cycle and determine, or generate, a composite heart failure metric 373 based on the plurality of generated heart failure metrics over x cardiac cycles. As described herein, the composite heart failure metric may be an average or median of the plurality of heart failure metrics. Additionally, as described herein, x, which represents the number of cardiac cycles for which the heart failure metric should be generated for, may be between 1 and about 30.

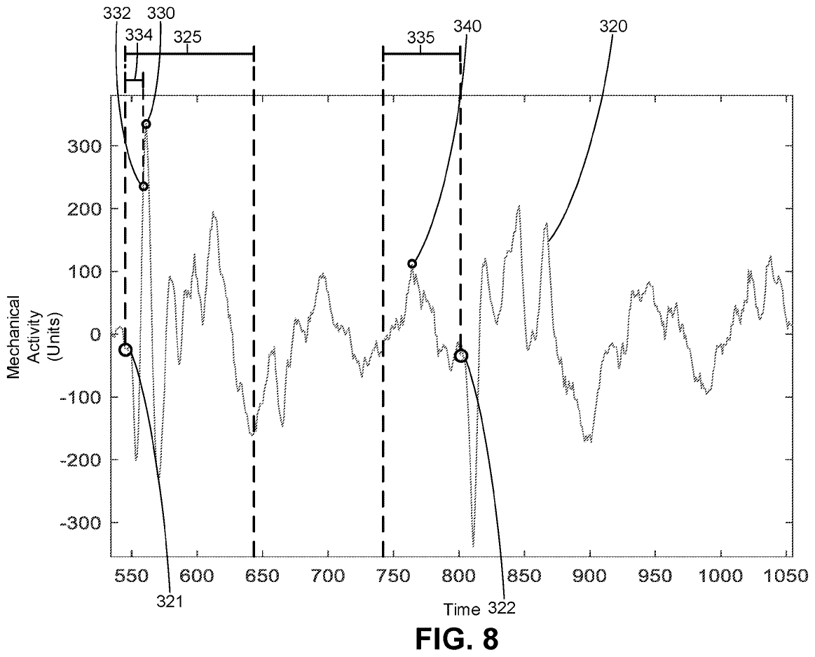

The plurality of heart failure metrics generated 306 in method 300 may include one or more of an electromechanical activation time interval, a late maximum value, and an early/late ratio between the late maximum value and an early maximum value, each of which will be described within respect to FIG. 8 that depicts an illustrative graph of a mechanical heart activity signal 320 over two cardiac cycles illustrating heart failure metrics. In one embodiment, the heart activity signal 320 may be expressed in terms of units as illustrative heart mechanical activity sensors may provide, or return, a signal based on the lowest resolution provided by an analog-to-digital convertor (ADC) provided therein. In one embodiment, the heart activity signal 320 may be expressed in terms of millivolts (mV).

The electromechanical activation time interval 334 is the time duration, or period, between a ventricular electrical activation event 321, whether intrinsic or paced, and a ventricular mechanical activation event 332 when the mechanical activity is initially greater than or equal to a selected percentage of an early maximum value 330. The early maximum value 330 may be the maximum value of the mechanical heart activity signal 320 within an early monitoring window 325 following the ventricular electrical activation event 321.

The early monitoring window 325 may be a selected percentage of a cardiac cycle such as, for example, between about 15% and about 55% of a cardiac cycle interval. The cardiac cycle interval may be defined as the time, or duration, of an entire cardiac cycle starting from a first ventricular electrical activation event 321 and ending at a second, next ventricular electrical activation event 322. In other words, the cardiac cycle interval may be between, or span, a first ventricular electrical activation event 321 and a second ventricular electrical activation event 322 following the first ventricular electrical activation event 321.

In one embodiment, the early monitoring window 325 is 40% of the cardiac cycle interval. In one or more other embodiments, the early monitoring window 325 may be greater than or equal to 15% of the cardiac cycle interval, greater than or equal to 20% of the cardiac cycle interval, greater than or equal to 25% of the cardiac cycle interval, greater than or equal to 30% of the cardiac cycle interval, greater than or equal to 35% of the cardiac cycle interval, or greater than or equal to 37% of the cardiac cycle interval, and/or less than or equal to 55% of the cardiac cycle interval, less than or equal to 50% of the cardiac cycle interval, less than or equal to 45% of the cardiac cycle interval, or less than or equal to 43% of the cardiac cycle interval.

As described herein, the electromechanical activation time interval 334 utilizes the ventricular mechanical activation event 332 when the mechanical activity is initially greater than or equal to a selected percentage of the early maximum value 330. The selected percentage may provide a consistent fiducial representative of the ventricular mechanical activation event 332, which starts, or initiates, prior to the early maximum value 330 of the mechanical heart activity signal 320. The selected percentage of the early maximum value 330 may be between about 50% and 95% of the early maximum value 330. In one embodiment and as shown in FIG. 8, the selected percentage of the early maximum value 330 is 70%. In one or more other embodiments, the selected percentage of the early maximum value 330 is greater than or equal to 50%, greater than or equal to 60%, greater than or equal to 70%, or greater than or equal to 75%, and/or less than or equal to 95%, less than or equal to 85%, or less than or equal to 80%. Additionally, in one embodiment, the ventricular mechanical activation event 332 may be the same as the early maximum value 330 (in this case, the selected percentage would be 0%).

The late maximum value 340 of the mechanical heart activity signal 320 may be determined within a late monitoring window 335 preceding the second ventricular electrical activation event 322 following the first ventricular electrical activation event 321. The late monitoring window 335 may be a selected percentage of a cardiac cycle such as, for example, between about 5% and about 40% of a cardiac cycle interval. In one embodiment, the late monitoring window 335 is 20% of the cardiac cycle interval. In one or more other embodiments, the late monitoring window 335 may be greater than or equal to 5% of the cardiac cycle interval, greater than or equal to 10% of the cardiac cycle interval, greater than or equal to 15% of the cardiac cycle interval, or greater than or equal to 20% of the cardiac cycle interval and/or less than or equal to 40% of the cardiac cycle interval, less than or equal to 35% of the cardiac cycle interval, less than or equal to 30% of the cardiac cycle interval, or less than or equal to 35% of the cardiac cycle interval.

The early/late ratio may be a ratio between, or of, the late maximum value 340 and the early maximum value 330. In other words, the late maximum value 340 may be divided by the early maximum value 330 resulting in the early/late ratio.

The method 300 may utilize the determined heart failure metrics to determine, or generate, a heart failure risk score 308. In other words, the heart risk score may be determined 308 based on the determined plurality of heart failure metrics. As described herein, with respect to FIG. 7, each of the heart failure metrics may be a composite heart failure metric generated, or based, on a plurality of heart failure metrics over a plurality of cardiac cycles. For instance, the electromechanical activation time interval may be determined over a plurality of cardiac cycles, such as three cardiac cycles, and a composite electromechanical activation time interval (e.g., mean or median) may be generated based on the plurality of determined electromechanical activation time intervals for the plurality of cardiac cycles, and the heart failure risk score may be generated based on the composite electromechanical activation time interval.

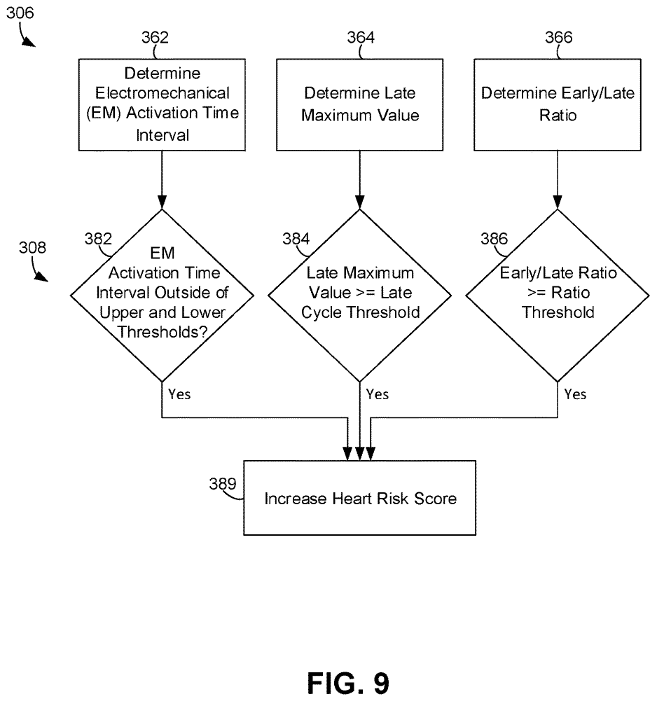

An illustrative method of determining heart failure risk metrics 306 and heart failure risk score 308 is depicted in FIG. 9. As shown, an electromechanical interval, late maximum value, and early/late ratio may be determined 362, 364, 366.

The heart risk score may be initialized, or started, as zero. To generate, or determine, the heart risk score based on the heart failure metrics, 308, each of the heart failure metrics may be compared, for example, to one or more corresponding thresholds or measurements. If the heart failure metric is indicative of heart failure or heart failure risk increase based on such one or more corresponding thresholds or other measurements, then the heart risk score, which was initialized to zero, may be increased by one 389.

More specifically, for example, lower and upper thresholds may be utilized when evaluating the determined electromechanical interval. For instance, if the electromechanical interval is between a lower threshold and an upper threshold 382, then the heart score may not be increased by one, and if the electromechanical interval is not between (e.g., falls outside of) the lower threshold and the upper threshold 382, then the heart score may be increased by one 389. In other words, if the electromechanical activation time interval is determined to be less than or equal to a lower electromechanical activation time threshold or greater than or equal to an upper electromechanical activation time threshold, then the heart risk score may be increased by one 389.

The lower electromechanical activation time threshold may be between about 10 milliseconds (ms) and about 300 ms. In one embodiment, the lower electromechanical interval activation time threshold is 170 milliseconds. The upper electromechanical activation time threshold may be between about 350 ms and about 1000 ms. In one embodiment, the upper electromechanical interval activation time threshold is 400 milliseconds. In other embodiments, the lower and upper electromechanical activation time thresholds may be expressed in terms of a percentage of cardiac cycle interval.

Further, for example, a late cycle threshold may be utilized when evaluating the determined late maximum value. For instance, if the late maximum value is greater than or equal to the late cycle threshold 384, then the heart score may be increased by one 389, and if the late maximum value is not greater than or equal to (or less than) the late cycle threshold 384, then the heart score may not be increased by one. In other words, if the late maximum value is determined to be greater than or equal to the late cycle threshold 384, then the heart risk score may be increased by one 389. The late cycle threshold may be between about 50 units and about 100 units. In one embodiment, the late cycle threshold is 88 units. The late cycle threshold may be between about 50 millivolts (mV) and about 25 mV. In one embodiment, the late cycle threshold is 44 mV.

Still further, for example, an early/late cycle ratio threshold may be utilized when evaluating the determined early/late ratio. For instance, if the early/late ratio is greater than or equal to the early/late ratio threshold 386, then the heart score may be increased by one 389, and if the early/late ratio is not greater than or equal to (or less than) the early/late ratio threshold 386, then the heart score may not be increased by one. The early/late ratio may be between about 0.1 and about 0.8. In one embodiment, the early/late ratio threshold is 0.5.

As a result, the heart risk score may be determined 308 to be between zero and three, in this embodiment. The method 300 of FIG. 6 may further include determining whether a patient has a heart failure risk or a patient has an increase in heart failure risk based on one or more heart risk scores 310, and if so, delivering, or transmitting, an alert 312 to one or more of the patient, the patient's clinician, and external care management system. For example, if the heart risk score is greater than or equal to a heart risk threshold, then it may be determined that the patient has a heart failure risk or has an increase in heart failure risk 310, and thus, an alert may be delivered, or transmitted, 312 to one or more of the patient, the patient's clinician, and external care management system.

Further, a heart risk score may be generated periodically, such as once day. In one or more embodiments, the heart risk score may be generated from mechanical and electrical heart activity monitored during the same time of each day such, for example, while the patient is at rest after waking up in the morning when the heart rate corresponds to baseline resting levels (e.g., less than 80 beats per minute) or in the middle of night when the heart rate corresponds to baseline resting levels (e.g., less than 80 beats per minute). In this example, it may not be determined that the patient is at heart failure risk or at an increased heart failure risk unless the heart failure risk score risk score has been greater or equal to the heart risk threshold for a number of consecutive, or successive, days 310. Thus, if the heart risk score is greater than or equal to a heart risk threshold for a selected number of consecutive, or successive, days, then it may be determined that the patient has a heart failure risk or has an increase in heart failure risk 310, and thus, an alert may be delivered, or transmitted, 312 to one or more of the patient, the patient's clinician, and external care management system. Additionally, as opposed to utilizing consecutive days, the determination 310 may determine, when the heart risk score is greater than or equal to a heart risk threshold for a first selected number of days (e.g., 5 days) out of a second selected number of days (e.g., 7 days), that the patient has a heart failure risk or has an increase in heart failure risk 310, and thus, an alert may be delivered, or transmitted, 312 to one or more of the patient, the patient's clinician, and external care management system. In other words, if the heart failure risk score provides a trend that the patient is at heart failure risk or at an increased heart failure risk 310, then an alert may be transmitted or delivered 312.

Further, the heart failure risk score may be utilized by external systems as one factor, metric, or piece of data for use in deriving a composite heart risk score. For example, one or more of the external device 192 and computing devices 194n may utilize the heart failure risk score in conjunction with one or more other one or more diagnostic parameters to determine, or derive, a composite heart risk score. In at least one embodiment, the heart risk score may be transmitted, or delivered, to an external system, and the external system may be configured to derive a composite heart risk score using a Bayesian approach based on the received heart risk score and one or more diagnostic parameters. Illustrative systems that may use one or more heart failure metrics and heart failure risk score as described herein may be described in U.S. Pat. App. Pub. No. 2022/0193419 entitled “Method and Apparatus for Monitoring Tissue Fluid Content for use in an Implantable Cardiac Device” and published on Jun. 23, 2022, which is incorporated by reference herein in its entirety.

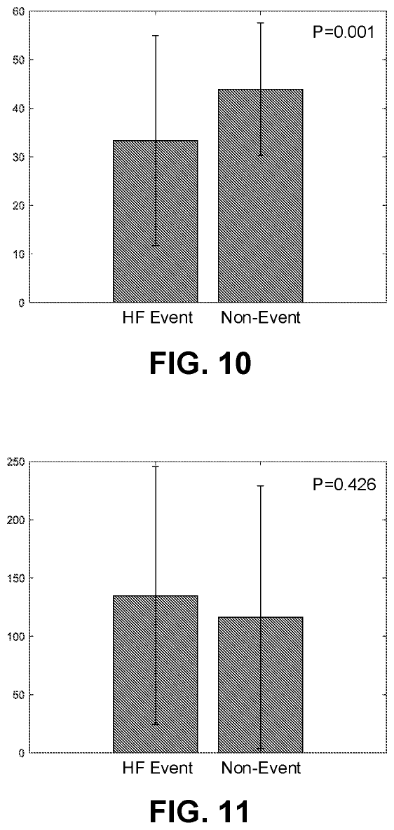

Comparative bar graphs showing the correlation between various metrics including heart failure metrics and heart failure risk score and patients that experienced heart failure events and to patients that did not experience heart failure events are shown in FIGS. 10-14. In particular, the bar graphs were generated from data gathered from heart sounds recorded from piezoelectric sensors incorporated within, or part of, cardiac resynchronization devices implanted in heart failure patients treated with cardiac resynchronization therapy. As shown, the differences in electromechanical intervals being indicative of increased heart failure risk and patients that experienced heart failure events and for patients that did not experience heart failure events are depicted in FIG. 10. The p-value is 0.001, which demonstrates that the differences in electromechanical interval is significant, hence suggesting that too short of an electromechanical interval is associated withs increased heart failure risk.

As shown, the differences in late maximum value of mechanical activity being indicative of increased heart failure risk between patients that experienced heart failure events and patients that did not experience heart failure events is depicted in FIG. 11. Though the differences are not significant (p=0.426) there is a trend towards higher amplitudes in patients experiencing heart failure events, which indicates that the late maximum value of mechanical activity may be associated with an increased heart failure risk.

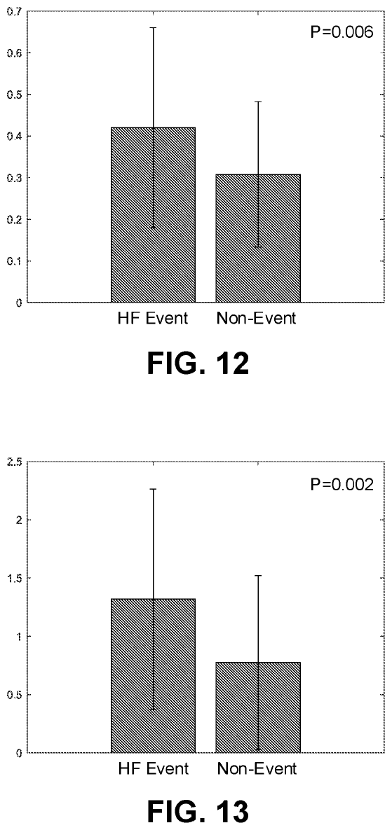

Further, as shown, the differences in early/late ratio of mechanical activity being indicative of increased heart failure risk between patients that experienced heart failure events and patients that did not experience heart failure events is depicted in FIG. 12. The p-value is 0.006, which demonstrates significant differences and suggests that that a higher value of the early/late ratio of mechanical activity is associated with an increased heart failure risk.

Still further, as shown, the differences in heart sounds-based heart risk score for patients being indicative of increased heart failure risk for patients that experienced heart failure events and for patients that did not experience heart failure events is depicted in FIG. 13. The p-value of 0.006 shows statistical significance and suggests that an increased risk score is associated with heart failure-related mortality and hospitalizations.

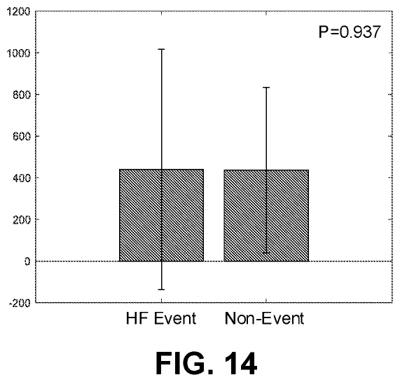

Also, as shown, the differences in early maximum value, or S1 amplitude, being indicative of increased heart failure risk for patients that experienced heart failure events and for patients that did not experience heart failure events is depicted in FIG. 14. The p-value is 0.937, which indicates a lack of statistical significance and that there is not much difference in early maximum value, or S1 amplitude, between patients who had heart failure events versus those who did not have heart failure events.

As a result, each of the heart failure metrics described herein such as the electromechanical interval, the late maximum value, and the early/late ratio substantially better correlate to an increased heart failure risk than the early maximum value. Additionally, the heart failure risk score also substantially better correlates to an increased heart failure risk than the early maximum value and further provides a more robust valuation of an increased heart failure risk as the heart failure risk score is based on more than a single metric.

### EXAMPLES

Example Ex1: A device comprising:
a mechanical heart activity sensor configured to monitor mechanical activity of a patient's heart; and
a computing apparatus comprising processing circuitry and operably coupled to the mechanical heart activity sensor, the computing apparatus configured to:
   monitor mechanical activity of the patient's heart using the mechanical heart activity sensor;
   determine a plurality of heart failure metrics for one or more cardiac cycles using the monitored mechanical activity, wherein the plurality of heart failure metrics comprises an electromechanical activation time interval, a late maximum value, and an early/late ratio between the late maximum value and an early maximum value, determining the plurality of heart failure metrics for each of the one or more cardiac cycles comprises:
      determining the early maximum value of the mechanical activity within an early monitoring window following a first ventricular electrical activation event;
      determining the electromechanical activation time interval based on the first ventricular electrical activation event and the early maximum value;
      determining the late maximum value of the mechanical activity within a late monitoring window preceding a second ventricular electrical activation event following the first ventricular electrical activation event; and
      determining the early/late ratio between the late maximum value and the early maximum value; and
   determine a heart failure risk score based on the determined plurality of heart failure metrics for the one or more cardiac cycles.
Example Ex2: A method comprising:
monitoring mechanical activity of a patient's heart;
determining a plurality of heart failure metrics for one or more cardiac cycles using the monitored mechanical activity, wherein the plurality of heart failure metrics comprises an electromechanical activation time interval, a late maximum value, and an early/late ratio between the late maximum value and an early maximum value, determining the plurality of heart failure metrics for each of the one or more cardiac cycles comprises:
   determining the early maximum value of the mechanical activity within an early monitoring window following a first ventricular electrical activation event;
   determining the electromechanical activation time interval based on the first ventricular electrical activation event and the early maximum value;
   determining the late maximum value of the mechanical activity within a late monitoring window preceding a second ventricular electrical activation event following the first ventricular electrical activation event; and
   determining the early/late ratio between the late maximum value and the early maximum value; and
determine a heart failure risk score based on the determined plurality of heart failure metrics for the one or more cardiac cycles.
Example Ex3: The device as in Example Ex1 or the method as in Example Ex2, wherein the mechanical heart activity sensor comprises one or more of a piezoelectric sensor to monitor vibration of the heart, a motion sensor to monitor heart motion, and a microphone to monitor heart sounds.
Example Ex4: The device or method as in any one of Examples Ex1-Ex3, wherein the determining the electromechanical activation time interval based on the first ventricular electrical activation event and the early maximum value comprises:
determining a ventricular mechanical activation event when the mechanical activity is initially greater than or equal to a selected percentage of the early maximum value; and
determining the electromechanical activation time interval between the first ventricular electrical activation event and the ventricular mechanical activation event.
Example Ex5: The device of method as in Example Ex4, wherein the selected percentage is greater than or equal to 70%.
Example Ex6: The device or method as in any one of Examples Ex1-Ex5, wherein the early monitoring window is greater than equal to 40% of a cardiac cycle interval between the first ventricular electrical activation event and the second ventricular electrical activation event.
Example Ex7: The device or method as in any one of Examples Ex1-Ex5, wherein the late monitoring window is greater than equal to 20% of a cycle interval between the first ventricular electrical activation event and the second ventricular electrical activation event.
Example Ex8: The device or method as in any one of Examples Ex1-Ex7, wherein the one or more cardiac cycles comprises a plurality of cardiac cycles, wherein determining the plurality of heart failure metrics for one or more cardiac cycles using the monitored mechanical activity comprises:
determining the plurality of heart failure metrics for each of the plurality of cardiac cycles; and
determining a composite heart failure metric for each different heart failure metric based on the plurality of corresponding heart failure metrics, and
wherein determining a heart failure risk score based on the determined plurality of heart failure metrics for the one or more cardiac cycles comprises determining the heart failure risk score based on the determined composite heart failure metrics.
Example Ex9: The device of method as in Example Ex8, wherein the composite heart failure metric is a median.
Example Ex10: The device or method as in any one of Examples Ex8-Ex9, wherein plurality of cardiac cycles comprises five or more cardiac cycles.
Example Ex11: The device or method as in any one of Examples Ex1-Ex10, wherein determining the heart failure risk score based on the determined plurality of heart failure metrics for the one or more cardiac cycles comprises:
determining that the electromechanical activation time interval is less than or equal to a lower electromechanical activation time threshold or greater than or equal to an upper electromechanical activation time threshold; and
increasing the heart failure risk score in response to determination that that the electromechanical activation time interval is less than or equal to the lower electromechanical activation time threshold or greater than or equal to the upper electromechanical activation time threshold.
Example Ex12: The device or method as in Example Ex11, wherein the lower electromechanical activation time threshold is greater than or equal to 170 milliseconds and the upper electromechanical activation time threshold is less than or equal to 400 milliseconds.
Example Ex13: The device or method as in any one of Examples Ex1-Ex12, wherein determining the heart failure risk score based on the determined plurality of heart failure metrics for the one or more cardiac cycles comprises:
determining that the late maximum value greater than or equal to a late cycle threshold; and
increasing the heart failure risk score in response to determination that the late maximum value is greater than or equal to the late cycle threshold.
Example Ex14: The device or method as in any one of Examples Ex1-Ex13, wherein determining the heart failure risk score based on the determined plurality of heart failure metrics for the one or more cardiac cycles comprises:
determining that the early/late ratio between the late maximum value and the early maximum value greater than or equal to an early/late ratio threshold; and
increasing the heart failure risk score in response to determination that the early/late ratio between the late maximum value and the early maximum value greater than or equal to the early/late ratio threshold.
Example Ex15: The device or method as in Example Ex14, wherein the early/late ratio threshold is greater than or equal to 0.5.
Example Ex16: The device or method as in any one of Examples Ex1-Ex15, wherein determining the heart failure risk score based on the determined plurality of heart failure metrics for the one or more cardiac cycles comprises periodically, once a day, determining the heart failure risk score based on the determined plurality of heart failure metrics for the one or more cardiac cycles.
Example Ex17: The device or method as in Example Ex16, wherein the computing apparatus is further configured to execute or the method further comprises:
determining that the heart failure risk score is greater than or equal to a heart risk threshold for more than a number of successive days; and
transmitting an alert in response to determining that the heart failure risk score is greater than or equal to the heart risk threshold for more than the number of successive days.
Example Ex18: The device as in any one of Examples Ex1-Ex17, wherein the computing apparatus is further configured to transmit the heart failure risk score to an external system, the external system configured to derive a composite heart failure risk score using a Bayesian approach based on the received heart failure risk score and one or more diagnostic parameters.
Example Ex19: The method as in any one of Examples Ex1-Ex17, the method further comprising deriving a composite heart failure risk score using a Bayesian approach based on the heart failure risk score and one or more diagnostic parameters.
Example Ex20: A system comprising:
a remote computing apparatus comprising processing circuitry and configured to: receive mechanical heart activity of a patient;
   determine a plurality of heart failure metrics for one or more cardiac cycles using received mechanical heart activity, wherein the plurality of heart failure metrics comprises an electromechanical activation time interval, a late maximum value, and an early/late ratio between the late maximum value and an early maximum value, determining the plurality of heart failure metrics for each of the one or more cardiac cycles comprises:
      determining the early maximum value of the mechanical heart activity within an early monitoring window following a first ventricular electrical activation event;
      determining the electromechanical activation time interval based on the first ventricular electrical activation event and the early maximum value;
      determining the late maximum value of the mechanical heart activity within a late monitoring window preceding a second ventricular electrical activation event following the first ventricular electrical activation event; and
      determining the early/late ratio between the late maximum value and the early maximum value; and
   determine a heart failure risk score based on the determined plurality of heart failure metrics for the one or more cardiac cycles.
Example Ex21: The system of Example Ex20, the system further comprising:
one or more medical devices, each medical device comprising a mechanical heart activity sensor configured to monitor mechanical heart activity of a patient's heart and configured to monitor and transmit the mechanical heart activity of the patient's heart using the mechanical heart activity sensor to the remote computing apparatus.
Example Ex22: The system as in any one of Examples Ex20-Ex21, wherein the remote computing apparatus is further configured to derive a composite heart failure risk score using a Bayesian approach based on the determined heart failure risk score and one or more diagnostic parameters.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described methods, processes, and techniques, including those attributed to the IMD 16, the programmer 24, the external device 192, and computing devices 194n, may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. The terms “computing apparatus,” “controller” “module,” “processor,” or “processing circuitry” may generally refer to any of the foregoing logic circuitry, alone or in combination with other logic circuitry, or any other equivalent circuitry. Also, the techniques could be fully implemented in one or more circuits or logic elements. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

All references and publications cited herein are expressly incorporated herein by reference in their entirety for all purposes, except to the extent any aspect directly contradicts this disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

The singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

1. A device comprising:
a mechanical heart activity sensor configured to monitor mechanical activity of a patient's heart; and
a computing apparatus comprising processing circuitry and operably coupled to the mechanical heart activity sensor, the computing apparatus configured to:
monitor mechanical activity of the patient's heart using the mechanical heart activity sensor;
determine a plurality of heart failure metrics for one or more cardiac cycles using the monitored mechanical activity, wherein the plurality of heart failure metrics comprises an electromechanical activation time interval, a late maximum value, and an early/late ratio between the late maximum value and an early maximum value, wherein determining the plurality of heart failure metrics for each of the one or more cardiac cycles comprises:
determining the early maximum value of the mechanical activity within an early monitoring window following a first ventricular electrical activation event;
determining the electromechanical activation time interval based on the first ventricular electrical activation event and the early maximum value;
determining the late maximum value of the mechanical activity within a late monitoring window preceding a second ventricular electrical activation event following the first ventricular electrical activation event; and
determining the early/late ratio between the late maximum value and the early maximum value; and
determine a heart failure risk score based on the determined plurality of heart failure metrics for the one or more cardiac cycles.

2. (canceled)

3. A system comprising:
a remote computing apparatus comprising processing circuitry and configured to:
receive mechanical heart activity of a patient from a mechanical heart activity sensor;
determine a plurality of heart failure metrics for one or more cardiac cycles using received mechanical heart activity, wherein the plurality of heart failure metrics comprises an electromechanical activation time interval, a late maximum value, and an early/late ratio between the late maximum value and an early maximum value, wherein determining the plurality of heart failure metrics for each of the one or more cardiac cycles comprises:
determining the early maximum value of the mechanical heart activity within an early monitoring window following a first ventricular electrical activation event;
determining the electromechanical activation time interval based on the first ventricular electrical activation event and the early maximum value;
determining the late maximum value of the mechanical heart activity within a late monitoring window preceding a second ventricular electrical activation event following the first ventricular electrical activation event; and
determining the early/late ratio between the late maximum value and the early maximum value; and
determine a heart failure risk score based on the determined plurality of heart failure metrics for the one or more cardiac cycles.

4. The device as in claim 1, wherein the mechanical heart activity sensor comprises one or more of a piezoelectric sensor to monitor vibration of the heart, a motion sensor to monitor heart motion, and a microphone to monitor heart sounds.

5. The device of claim 1, wherein the determining the electromechanical activation time interval based on the first ventricular electrical activation event and the early maximum value comprises:
determining a ventricular mechanical activation event when the mechanical activity is initially greater than or equal to a selected percentage of the early maximum value; and
determining the electromechanical activation time interval between the first ventricular electrical activation event and the ventricular mechanical activation event.

6. The device of claim 1, wherein the early monitoring window is greater than equal to 40% of a cardiac cycle interval between the first ventricular electrical activation event and the second ventricular electrical activation event.

7. The device of claim 1, wherein the late monitoring window is greater than equal to 20% of a cycle interval between the first ventricular electrical activation event and the second ventricular electrical activation event.

8. The device of claim 1, wherein the one or more cardiac cycles comprises a plurality of cardiac cycles,
wherein determining the plurality of heart failure metrics for one or more cardiac cycles using the monitored mechanical activity comprises:
determining the plurality of heart failure metrics for each of the plurality of cardiac cycles; and
determining a composite heart failure metric for each different heart failure metric based on the plurality of corresponding heart failure metrics determined over the plurality of cardiac cycles,
wherein determining a heart failure risk score based on the determined plurality of heart failure metrics for the one or more cardiac cycles comprises determining the heart failure risk score based on the determined composite heart failure metrics.

9. The device of claim 8, wherein plurality of cardiac cycles comprises five or more cardiac cycles.

10. The device of claim 1, wherein determining the heart failure risk score based on the determined plurality of heart failure metrics for the one or more cardiac cycles comprises:
determining that the electromechanical activation time interval is less than or equal to a lower electromechanical activation time threshold or greater than or equal to an upper electromechanical activation time threshold; and
increasing the heart failure risk score in response to determination that that the electromechanical activation time interval is less than or equal to the lower electromechanical activation time threshold or greater than or equal to the upper electromechanical activation time threshold.

11. The device of claim 1, wherein determining the heart failure risk score based on the determined plurality of heart failure metrics for the one or more cardiac cycles comprises:
determining that the late maximum value greater than or equal to a late cycle threshold; and
increasing the heart failure risk score in response to determination that the late maximum value is greater than or equal to the late cycle threshold.

12. The device of claim 1, wherein determining the heart failure risk score based on the determined plurality of heart failure metrics for the one or more cardiac cycles comprises:
determining that the early/late ratio between the late maximum value and the early maximum value greater than or equal to an early/late ratio threshold; and
increasing the heart failure risk score in response to determination that the early/late ratio between the late maximum value and the early maximum value greater than or equal to the early/late ratio threshold.

13. The device of claim 1, wherein determining the heart failure risk score based on the determined plurality of heart failure metrics for the one or more cardiac cycles comprises periodically, once a day, determining the heart failure risk score based on the determined plurality of heart failure metrics for the one or more cardiac cycles.

14. The device of claim 1, wherein the computing apparatus is further configured to execute:
determining that the heart failure risk score is greater than or equal to a heart risk threshold for more than a number of successive days; and
transmitting an alert in response to determining that the heart failure risk score is greater than or equal to the heart risk threshold for more than the number of successive days.

15. (canceled)

16. A method comprising:
monitoring mechanical activity of a patient's heart from a mechanical heart activity sensor;
determining a plurality of heart failure metrics for one or more cardiac cycles using the monitored mechanical activity, wherein the plurality of heart failure metrics comprises an electromechanical activation time interval, a late maximum value, and an early/late ratio between the late maximum value and an early maximum value, wherein determining the plurality of heart failure metrics for each of the one or more cardiac cycles comprises:
determining the early maximum value of the mechanical activity within an early monitoring window following a first ventricular electrical activation event;
determining the electromechanical activation time interval based on the first ventricular electrical activation event and the early maximum value;
determining the late maximum value of the mechanical activity within a late monitoring window preceding a second ventricular electrical activation event following the first ventricular electrical activation event; and
determining the early/late ratio between the late maximum value and the early maximum value; and
determine a heart failure risk score based on the determined plurality of heart failure metrics for the one or more cardiac cycles.

17. The method of claim 16, wherein the mechanical heart activity sensor comprises one or more of a piezoelectric sensor to monitor vibration of the heart, a motion sensor to monitor heart motion, and a microphone to monitor heart sounds.

18. The method of claim 16, wherein the determining the electromechanical activation time interval based on the first ventricular electrical activation event and the early maximum value comprises:
determining a ventricular mechanical activation event when the mechanical activity is initially greater than or equal to a selected percentage of the early maximum value; and
determining the electromechanical activation time interval between the first ventricular electrical activation event and the ventricular mechanical activation event.

19. The method of claim 16, wherein the early monitoring window is greater than equal to 40% of a cardiac cycle interval between the first ventricular electrical activation event and the second ventricular electrical activation event.

20. The method of claim 16, wherein the late monitoring window is greater than equal to 20% of a cycle interval between the first ventricular electrical activation event and the second ventricular electrical activation event.

21. The method of claim 16, wherein the one or more cardiac cycles comprises a plurality of cardiac cycles,
wherein determining the plurality of heart failure metrics for one or more cardiac cycles using the monitored mechanical activity comprises:
determining the plurality of heart failure metrics for each of the plurality of cardiac cycles; and
determining a composite heart failure metric for each different heart failure metric based on the plurality of corresponding heart failure metrics determined over the plurality of cardiac cycles, and
wherein determining a heart failure risk score based on the determined plurality of heart failure metrics for the one or more cardiac cycles comprises determining the heart failure risk score based on the determined composite heart failure metrics.

22. The method of claim 21, wherein plurality of cardiac cycles comprises five or more cardiac cycles.

23. The method of claim 16, wherein determining the heart failure risk score based on the determined plurality of heart failure metrics for the one or more cardiac cycles comprises:
determining that the electromechanical activation time interval is less than or equal to a lower electromechanical activation time threshold or greater than or equal to an upper electromechanical activation time threshold; and
increasing the heart failure risk score in response to determination that that the electromechanical activation time interval is less than or equal to the lower electromechanical activation time threshold or greater than or equal to the upper electromechanical activation time threshold.

24. The method of claim 16, wherein determining the heart failure risk score based on the determined plurality of heart failure metrics for the one or more cardiac cycles comprises:
determining that the late maximum value greater than or equal to a late cycle threshold; and
increasing the heart failure risk score in response to determination that the late maximum value is greater than or equal to the late cycle threshold.

25. The method of claim 16, wherein determining the heart failure risk score based on the determined plurality of heart failure metrics for the one or more cardiac cycles comprises:
determining that the early/late ratio between the late maximum value and the early maximum value greater than or equal to an early/late ratio threshold; and
increasing the heart failure risk score in response to determination that the early/late ratio between the late maximum value and the early maximum value greater than or equal to the early/late ratio threshold.

26. The method of claim 16, wherein determining the heart failure risk score based on the determined plurality of heart failure metrics for the one or more cardiac cycles comprises periodically, once a day, determining the heart failure risk score based on the determined plurality of heart failure metrics for the one or more cardiac cycles.

27. The method of claim 16, wherein the method further comprises:
determining that the heart failure risk score is greater than or equal to a heart risk threshold for more than a number of successive days; and
transmitting an alert in response to determining that the heart failure risk score is greater than or equal to the heart risk threshold for more than the number of successive days.

28. The method The method of claim 16, the method further comprising deriving a composite heart failure risk score using a Bayesian approach based on the heart failure risk score and one or more diagnostic parameters.
